(19) United States
(12) Patent Application Publication
(10) Pub. No.: US 2015/0086521 A1
(43) Pub. Date: Mar. 26, 2015

(54) Medicament for the Treatment of Acute Myeloid Leukemia (AML)

(71) Applicant: ERYTECH PHARMA, Lyon (FR)

(72) Inventor: Yann Godfrin, Lyon (FR)

(21) Appl. No.: 14/386,533

(22) PCT Filed: Mar. 21, 2013
(86) PCT No.: PCT/EP2013/055928

§ 371 Date: Sep. 19, 2014

Related U.S. Application Data
(60) Provisional application No. 61/613,660, filed on Mar. 21, 2012.

(51) Int. Cl. A61K 38/50; A61K 47/46

(52) U.S. Cl. CPC A61K 38/50; A61K 47/46; C12Y 305/01001
USPC 424/93.73; 424/499

(57) ABSTRACT

The present invention relates to the therapeutic treatment of Acute Myeloid Leukemia (AML). It concerns in particular a novel composition for the treatment of this cancer and an associated therapeutic treatment method. The invention concerns a suspension of erythrocytes encapsulating asparaginase as a medicament for treating Acute Myeloid Leukemia (AML). The invention also concerns a method for treating Acute Myeloid Leukemia (AML) comprising administering an efficient amount of a suspension of erythrocytes encapsulating asparaginase.

The present invention relates to the therapeutic treatment of Acute Myeloid Leukemia (AML). It concerns in particular a novel composition for the treatment of this cancer and an associated therapeutic treatment method.

AML is a heterogeneous clonal disorder of hematopoietic progenitor cells and the most common malignant myeloid disorder in adults. The median age at presentation for patients with AML is around 65 years.

For the last 30 years, L-asparaginase has held a key role in chemotherapy for Acute Lymphoblastic Leukemia (ALL). Currently, L-asparaginase is used during the induction phase of ALL treatment for children and young adults (<55 years).

In adults, Capizzi R. L. and White C. (The Yale Journal of Biology and Medicine 61 (1988) 11-22) have reported a significant benefit of L-asparaginase in AML in adult patients with refractory or first relapse AML. The patient received high dose cytarabine and 6,000 IU/m² asparaginase.

Okada S. et al. (British Journal of Haematology 2003, 123, 802-809) have explored the potential efficacy of L-asparaginase in vitro on different subtypes of childhood AML In conclusion, cells from AML types M1, M4 and M5 were relatively sensitive to L-asparaginase, with M1 cells being the more sensitive.

Rubnitz J. E. et al. (Blood 2009, 113, 21, 5083-5089) was concerned with treatment of acute mixed-lineage leukemia in children. They observed that patients who failed to achieve complete remission with AML-directed therapy could often be induced with a regimen of prednisone, vincristine and L-asparaginase. The authors propose that treatment for biphenotypic leukemia begin with one course of AML-type induction therapy, with a provision for a shift to lymphoid-type induction therapy with a glucocorticoid, vincristine and L-asparaginase if the patients responds poorly.

However, if current standard therapy for children and young adults may comprise the administration of L-asparaginase, the enzyme is administered late in the treatment, during a consolidation phase, especially during the third consolidation phase. In the end, L-asparaginase is never used in the induction phase in clinic for patients that have been just diagnosed (first treatment against AML).

In addition, standard therapy for older patient with AML has poor outcome. There is one case known of a 66-year-old Japanese woman with AML who was induced with L-asparaginase, vincristine and prednisolone and achieved complete remission. However, in the majority of cases, elderly patients are unfit to intensive chemotherapy, say can not undergo to intensive chemotherapy, and only palliative treatment is available.

Asparaginase is an enzyme produced from bacterial microorganisms (E. coli or Erwinia chrysanthemi) which has been used for about thirty years in anti-leukaemia chemotherapy. This enzyme hydrolyses and depletes asparagine, an amino acid essential for the production of the proteins necessary for cell life. Now, in contrast to normal cells, certain cancerous lymphoblastic cells do not have the capacity to produce their asparagine themselves and are dependent on extracellular sources for the synthesis of their proteins. Treatment with asparaginase deprives them of this essential constituent and thus leads to their death. This antimitotic agent is selective for tumour cells.

The undesirable effects associated with this enzyme are well known, the main ones being certain allergies with clinical symptoms, diabetes and pancreatitis, mental disorders and coagulation disorders. In particular, natural asparaginase induces the production of circulating antibodies causing an increase in the clearance of asparaginase, and allergic reactions, sometimes very severe. Moreover, the short half-life of the enzyme (24 hrs) necessitates repeated injections and hospitalizations. This led to the development of a pegylated form, PEG-asparaginase, which has been approved by the FDA for first-line treatment of acute lymphoblastic leukaemia (ALL). In the end, the induction of antibodies has been observed with the three forms of asparaginase (E. coli, Erwinia and PEG-asparaginase), although the PEG form seems to be the least immunogenic. By reason of the premature stoppage of the treatment following allergic reactions, the therapeutic purpose of the asparaginase, which is to achieve a depletion of plasma asparagine for a defined period, is very often not attained.

The encapsulation of asparaginase in erythrocytes in order to improve its therapeutic index has been the subject of development studies. A tolerance study on asparaginase encapsulated in erythrocytes was under-taken by Kravtzoff et al. (C. Eur J Clin Pharmacol, 1996; 51(3-4): 221-5). Thirteen patients mostly suffering from non-Hodgkin lymphomas were given an injection of asparaginase encapsulated in erythrocytes (30 to 200 IU/kg). The study demonstrates an absence of allergic reaction compared to the direct injection of asparaginase (27%). In addition, the injection of asparaginase encapsulated in erythrocytes enables an asparagine depletion lasting for 50 consecutive days.

On the other hand, different studies (WO-A-2006/016247; Millan C G et al., Journal of Controlled Release, 2004, 95(1):27-49; Kravtzoff R et al., Journal of Pharmacy and Pharmacology, 1990, 42(7):473-476) describe the encapsulation of asparaginase in erythrocytes and the improvement of the pharmacokinetic properties of the encapsulated enzyme in the context of an application for lymphoma and acute lymphoblastic leukaemia.

In the end, there is a great need in finding an alternative to current treatments against AML, not only which could be beneficial for children and young adults, which are already eligible for intensive chemotherapy, but also for unfit patients, especially the elderly, for which no intensive chemotherapy is possible at the present time.

The inventors have found that this goal may be achieved and such alternative be proposed, by using L-asparaginase encapsulated inside erythrocytes. In particular, this encapsulated form is administrable, especially infusible, under suspension form. It may be used at any stage of a chemotherapy treatment, including particularly at the induction phase in patients which undergo their first AML treatment or newly diagnosed AML patients. The inventors have also found that this treatment is eligible for patients unfit for intensive chemotherapy, including newly diagnosed AML unfit patients, especially elderly patients. Not only patients which could not be eligible for intensive chemotherapy could now be treated with an efficient chemotherapy, but also they may benefit from the administration of a very efficient molecule, L-asparaginase, that was previously avoided due to the high level of undesirable effects. The commercial GRASPA® product is an example of suspension of human erythrocytes encapsulating L-asparaginase that may be used to perform the present invention.

A first object of the invention is a suspension of erythrocytes encapsulating asparaginase as a medicament for treating Acute Myeloid Leukemia (AML).

A second object of the invention is the use of a suspension of erythrocytes encapsulating asparaginase for the preparation of a medicament for treating Acute Myeloid Leukemia (AML).

A third object of the invention is a method for treating Acute Myeloid Leukemia (AML) comprising administering an efficient amount of a suspension of erythrocytes encapsulating asparaginase.

The additional features and the various embodiments which will be now presented do apply to the first, second and third objects of the invention.

In an embodiment, the patient is an elderly. Typically, an elderly is a person over 65 years.

In another embodiment, the patient is an adult (below 65 years), a young adult (<55 years) or a child.

In an embodiment, any AML patient is treated at the exclusion of a FAB M3 subtype patient.

In an embodiment, a FAB M1 subtype patient is treated. In an embodiment, a FAB M4 subtype patient is treated. In an embodiment, a FAB M5 subtype patient is treated. In an embodiment, FAB M1, M4 and M5 subtype patients are treated. In other embodiments, FAB M1 and M4, M1 and M5, or M4 and M5 subtype patients are treated.

In an embodiment, patients having AML tumoral cells expressing a low level of Asparagine Synthetase (ASNS) are treated.

In an embodiment, the patient is one unfit for intensive chemotherapy. By “unfit for intensive chemotherapy”, it is meant a patient who does not support or is likely to not support the toxicity associated with the standard protocol of chemotherapy. Such patients are encountered in any population. It is more common in the elderly population, especially persons over 65 years.

Typically, the erythrocytes are in suspension in a pharmaceutically acceptable saline solution. This can be a standard medium for erythrocytes, in particular a solution of NaCl (preferably 0.9%) possibly with added ingredients such as glucose, dextrose, adenine and/or mannitol. Standard media that can be used are SAG mannitol and ADsol which are solutions based on adenine, glucose, mannitol and sodium chloride. The solution can further contain a preservative such as L-carnitine.

In an embodiment, one dose of suspension comprises from 50 to 500 IU, preferably from 50 to 200 IU, more preferably from 80 to 170 IU of encapsulated asparaginase per kg body weight. Typical doses are 100 IU and 150 IU of asparaginase per kg body weight. By definition, a dose is the amount of asparaginase administered to the patient at a given time.

Encapsulated means that the enzyme is contained inside the erythrocytes. It is possible however that some minor amount of asparaginase is retained within the erythrocyte wall.

Administration is preferably effected by intravenous or intra-arterial injection. In a convenient embodiment, administration is performed by perfusion from a blood bag or the like. Administration is typically effected intravenously into the arm or via a central catheter.

Typically one dose is perfused or infused and this may last from about 15 to 45 minutes.

In an embodiment, doses of suspensions are administered to the same patient with a lag time between two administrations. The lag time is generally above or equal to 14 days. It may be from 14 to 45 days. The longest lag times, of around 45 days, are especially adapted to patients having developed aplasia as a result of the treatment with the preceding dose or drug. The physician may monitor the end of aplasia and administer the dose of asparaginase after recovery of aplasia.

According to the invention, the suspension contains an amount of erythrocytes and an amount of encapsulated asparaginase that is sufficient to deliver to the patient the dose of asparaginase that has been decided. Typically, the suspension of the invention may contain between 30 and 300 IU of encapsulated asparaginase per ml, preferably between 70 and 150 IU per ml.

The suspension can be ready for use and have a haematocrit suitable for administration by injection or by perfusion without dilution.

In an embodiment, the suspension is ready for use. According to the invention, the haematocrit of the suspension ready for use advantageously lies between about 40 and about 70%, preferably between about 45 and about 55%, and better about 50%.

In another embodiment, the suspension has to be diluted before use, e.g. before administration by injection or by perfusion. In an embodiment of such a suspension to be diluted before use, the haematocrit before dilution lies between 60 and 90%.

The suspension is preferably packaged at a volume of about 10 to about 250 ml. The packaging is preferably in a blood bag of the type suitable for a blood transfusion. The whole of the quantity of encapsulated asparaginase corresponding to the medical prescription is preferably contained in one blood bag and the like. It may also be contained in several blood bags and the like.

In a very advantageous embodiment, the suspension of the invention is for use in first intention in a patient in need thereof. The patient may be one for which the AML diagnosis has just been made or is treated for the first time against AML. The patient may be also one relapsing or having relapsed. The use in first intention means that the suspension is used at the beginning of the treatment or the new treatment, during the induction phase (the first treatment phase which is designed to induce remission). The present invention allows one to use asparaginase in an intensive chemotherapy, with asparaginase administered at an early stage.

Specific embodiments are thus:
the suspension according to the invention is for use as a medicament during the induction phase of a treatment against AML;
the use of the suspension of the invention for the preparation of a medicament to be administered during the induction phase in a treatment against AML;
a method to treat AML comprising the administration of a suspension according to the invention during the induction phase of a treatment against AML.

Theses embodiments may be applied to any patient in need thereof, including very advantageously the unfit patients.

In a protocol which is beneficial for the patient, say induces remission, the induction phase may be followed by several consolidation phases, generally 2 or 3. The suspension according to the invention may be used at any time during a treatment protocol, i.e. at any of or all the induction and consolidation phases. In an embodiment, the suspension is used at all phases.

In an embodiment, the suspension is used as a medicament for treating Acute Myeloid Leukemia (AML) in a patient in a multi-therapy or combined therapy. This means that the suspension of erythrocyte encapsulating asparaginase is used within a chemotherapeutic protocol in which one or several other chemotherapeutic agents are used.

By another chemotherapeutic agent, it is meant any standard or new chemical or biological agent for the treatment of AML. Some examples include: cytarabine (e.g. Aracytine® or AraC), mitoxantrone, amsacrine, etoposide, thioguanine, prednisolone, vincristine, VP16, daunorubicine, azacitidine, decitabine.

In a given embodiment, said another chemotherapeutic agent is cytarabine. Cytarabine may be used at a low dose regimen or at a high dose regimen. By low dose, it is referred to the low dose regimen used in the standard protocols. The low dose is typically 10 or 20 mg/m², generally twice a day. By contrast, a high dose regimen is of the order of 200 mg/m²/d (d=day) or more. The low dose is defined herein with the range of from 1 to 100 mg/m²/d, in particular 5 to 50 mg/m²/d.

In an embodiment, cytarabine is administered daily, preferably during 5 to 15 contiguous days, especially during 8 to 12 days, for example 10 days.

In an embodiment, the method for treating Acute Myeloid Leukemia (AML) comprises administering an efficient amount of a suspension of erythrocytes encapsulating asparaginase, and comprises the following induction phase scheme:

1^{st} month

Cytarabine
1 to 100 mg/m²/d, in particular 5 to 50 mg/m²/d, e.g. 20, 30 or 40 mg/m²/d,
during 5 to 15 days, especially during 8 to 12 days e.g. 10 days, preferably at D1 to D10,

Suspension of erythrocytes encapsulating asparaginase
50 to 500 IU, preferably from 50 to 200 IU, more preferably from 80 to 170 IU of encapsulated asparaginase per kg body weight; typical doses are 100 IU and 150 IU
Administration of one dose after the last cytarabine administration,

2^{nd} month until the end of the induction phase, i.e. 12^{th} month, each month

Cytarabine
1 to 100 mg/m²/d, in particular 5 to 50 mg/m²/d, e.g. 20, 30 or 40 mg/m²/d,
during 5 to 15 days, especially during 8 to 12 days e.g. 10 days, preferably at D1 to D10,

Suspension of erythrocytes encapsulating asparaginase
50 to 500 IU, preferably from 50 to 200 IU, more preferably from 80 to 170 IU of encapsulated asparaginase per kg body weight; typical doses are 100 IU and 150 IU
Administration of one dose at D1, D2 or D3.

In an embodiment:

1^{st} 28 days period

Cytarabine 40 mg/m², e.g. 20 mg/m² bid (twice a day) D1 to D10, daily

One dose suspension of erythrocytes encapsulating asparaginase 100 IU/kg at D11

2^{nd} 28 days period until 12^{th} month

Cytarabine 40 mg/m², e.g. 20 mg/m² bid at D1 to D10, daily

One dose suspension of erythrocytes encapsulating asparaginase 100 IU/kg at D1.

In an embodiment, mitoxantrone is associated with the suspension and cytarabine during the same phase, especially the induction phase.

Asparaginase itself is designated by the CAS number: 9015-68-3. Its usual name is asparaginase; other common names for it are: colaspase, L-asparaginase and L-asparagine aminohydrolase.

The term asparaginase in the sense of the present invention covers asparaginase of any origin, it can in particular be of natural or recombinant origin, and any derivative incorporating asparaginase, such as for example a PEG form, or a fragment retaining the activity of L-asparaginase. It also covers asparaginase whatever its bacterial origin. Thus, the asparaginase may be of the E. coli type, in particular E. coli HAP-A-1-3, of the Erwinia chrysanthemi type or of the Wolinella succinogenes type. “Type” is understood to mean that it can be obtained from a culture of the bacterium in question or that it can be recombinant, in other words a form of asparaginase of that bacterium obtained by genetic engineering. In a preferred implementation mode, it is of the E. coli HAP-A-1-3 type.

The term asparaginase also covers asparaginase-like substances which in the sense of the invention are bacterial enzymes having an L-asparagine aminohydrolase activity. By way of example, Acinetobacter glutaminase asparaginase (AGA) may be cited.

The erythrocytes are preferably of human origin. In an embodiment, the erythrocytes comes from the patient itself.

The techniques enabling the encapsulation of active principles in erythrocytes are known and the basic technique by lysis-resealing, which is preferred here, is described in the patents EP-A-101 341 and EP-A-679 101, to which the person skilled in the art will be able to refer. According to this technique, the primary compartment of a dialysis unit (for example dialysis bag or dialysis cartridge) is continuously fed with a suspension of erythrocytes, whereas the secondary compartment contains an aqueous solution hypotonic relative to the suspension of erythrocytes in order to lyse the erythrocytes; next, in a resealing unit, the resealing of the erythrocytes is induced in the presence of asparaginase by increasing the osmotic and/or oncotic pressure, and then a suspension of erythrocytes containing asparaginase is collected.

Among the variations described up to the present, the method described in WO-A-2006/016247, which makes it possible to encapsulate asparaginase in an efficient, reproducible, reliable and stable manner, is preferred. This method comprises the following stages:
1—suspension of a erythrocytes pellet in an isotonic solution at a haematocrit level greater than or equal to 65%, refrigeration between +1 and +8° C.,
2—measurement of the osmotic fragility using a sample of erythrocytes from this same corpuscle pellet, it being possible to perform stages 1 and 2 in any order (including in parallel),
3—procedure of lysis and internalization of the asparaginase, within a same enclosure, at a temperature constantly maintained between +1 and +8° C., comprising the passage of the suspension of erythrocytes at a haematocrit level greater than or equal to 65% and of a hypotonic lysis solution refrigerated to between +1 and +8° C. in a dialysis cartridge, the lysis parameters being adjusted on the basis of the previously measured osmotic fragility; and
4—a resealing procedure carried out in a second enclosure in the interior of which the temperature lies between +30 and +40° C., and in the presence of a hypertonic solution.

“Internalization” is understood to mean penetration of the asparaginase into the interior of the erythrocytes.

In particular, for the dialysis, the erythrocyte pellet is suspended in an isotonic solution at a high haematocrit level, greater than or equal to 65%, and preferably greater than or equal to 70%, and this suspension is refrigerated to between +1 and +8° C., preferably between +2 and +6° C., typically around +4° C. According to a particular mode, the haematocrit level lies between 65 and 80%, preferably between 70 and 80%.

The osmotic fragility is advantageously measured on the erythrocytes just before the lysis stage, in the presence or absence of asparaginase in the suspension. The erythrocytes or the suspension containing them are advantageously at a temperature close to or identical to the temperature selected for the lysis. According to another advantageous characteristic of the invention, the measurement of osmotic fragility carried out is rapidly utilized, in other words the lysis procedure is carried out shortly after the sample is taken. Preferably, this time lapse between sampling and start of lysis is less than or equal to 30 minutes, better still less than or equal to 25 and even to 20 minutes.

For more details concerning the manner of operating the lysis-resealing procedure, with measurement and allowance for the osmotic fragility, the person skilled in the art will be able to refer to WO-A-2006/016247.

The present invention will now be described in more detail by means of implementation modes taken as non-limiting examples.

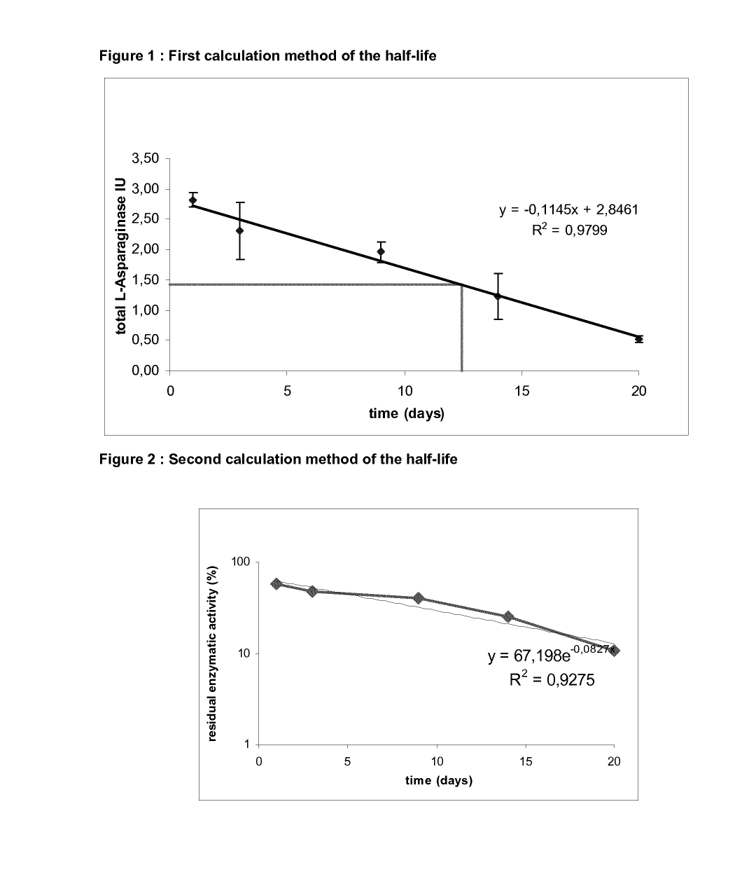

FIGS. 1 and 2 are graph illustrating the calculation methods of the half-life of Asparaginase or encapsulated Asparaginase.

### EXAMPLE 1

### Method for Encapsulation of L-Asparaginase in Murine Erythrocytes

The L-asparaginase (Kidrolase®, OPI-EUSA Limonest France) is encapsulated in murine erythrocytes (OF1 mice) by the method of hypotonic dialysis in a dialysis bag. The blood is centrifuged beforehand to remove the plasma, and then washed three times with 0.9% NaCl. The haematocrit is adjusted to 70% in the presence of the asparaginase, added to a final concentration of 400 IU/ml of erythrocytes or red blood cells (RBC) before starting the dialysis. The dialysis lasts 50 minutes at 4° C. against a lysis buffer of low osmolarity. The murine erythrocytes are then resealed through the addition of a high osmolarity solution and incubating 30 minutes at 37° C. After two washings with 0.9% NaCl and one washing with Sag-mannitol supplemented with bovine serum albumin BSA (6%), the erythrocytes are adjusted to haematocrit 50%. The erythrocytes encapsulating the L-asparaginase are called L-Aspa RBC. The encapsulation generates L-Aspa RBC at a concentration of 40 IU of asparaginase/ml of RC at 50% haematocrit.

During the encapsulation procedure, the whole blood, the washed RBC, the RBC mixed with the L-asparaginase (before dialysis) and the RBC loaded with L-asparaginase (after dialysis) are tested for:
haematocrit (Ht)
average corpuscular volume (ACV)
average corpuscular haemoglobin concentration (ACHC)
total haemoglobin concentration and
cell count.

Aliquots of the cell suspensions are withdrawn before and after the hypotonic dialysis for measurement of the L-asparaginase enzyme activity. The estimation of the L-asparaginase was performed according to the protocol published in: Orsonneau et al., Ann Biol Clin, 62: 568-572.

### EXAMPLE 2

### Determination of the Pharmacokinetic and Pharmacodynamic Parameters of L-Aspa RBC in the Mouse

Murine L-Aspa RBC were injected into OF1 mice so as to determine the half-life of the L-Aspa RBC in circulation in the mouse and to demonstrate the depletion of L-asparagine in mouse plasma. A single dose of 200 IU/kg was injected into each mouse by the intravenous route.

The half-life of the L-Aspa RBC is 12.39±0.74 days (calculation based on the activity of the enzyme). When the half-life of the murine L-Aspa RBC is calculated via cell labelling (CFSE-L-Aspa RBC), the value is 16.52±3.13 days, and 15.83±3.31 days for RBC simply labelled with CFDA-SE (CFSE RBC).

The depletion of plasma L-asparagine is total (<2 μM), and is obtained 15 minutes after injection of the L-Aspa RBC and persists for at least 20 days.

TABLE 1
Pharmacokinetic data obtained for L-Aspa RBC and
for murine RBC labelled with CFDA-SE (CFSE RBC)

| | RBC | | L-asparaginase | |
|---|---|---|---|---|
| | survival at | half-life | survival at | half-life |
| | 24 hrs (%) | (days) | 24 hrs (%) | (days) |
| L-Aspa RBC | — | — | 57.9 ± 2.5 | 12.39 ± 0.74 |
| CFSE-L-Aspa | 80.7 ± 0.7 | 16.52 ± 3.13 | 76.7 ± 1.4 | 12.20 ± 1.38 |
| RBC | | | | |
| CFSE RBC | 92.7 ± 2.6 | 15.83 ± 3.31 | — | — |

The half-life was calculated as follow:

The intercept point obtained from the plot equation is divided by two. Then the corresponding value of the abscissa is calculated tanks to the plot.

An example of the calculation is shown on FIG. 1, wherein the calculated intercept point is 2.8461.

Half of the intercept point: 1.42

Calculation of the corresponding value of the abscissa: 1.42=(−0.1145*X)+2.8 X=(1.42−2.8)/−0.1145=−1.38/−0.1145=12 days.

More real half-time could be calculated with a second method wherein the ordinate sale is a logarithm scale and the abscissa scale is a linear scale as shown on FIG. 2.

The half-time is calculated as follow:

Ln(2)/plot coefficient of the curve.

In the example of FIG. 2 (which is the same example as in FIG. 1) the half-time is:

Ln(2)/0.083=8.3 days.

TABLE 2
Measurement of residual L-asparaginase activity as
a function of time for L-Aspa RBC and free L-asparaginase
Time

| | 15 min | 24 hr | 3 d | 9 d | 14 d | 20 d | |
|---|---|---|---|---|---|---|---|
| L-Aspa | 100 | 57.1 | 46.9 | 39.8 | 24.9 | 10.6 | residual |
| RBC | | | | | | | asparaginase |
| Free L- | 100 | 3.3 | 0 | 0 | 0 | 0 | activity (%) |
| Aspa | | | | | | | |

Furthermore, estimation of the circulating plasma L-asparaginase shows that beyond 24 hours after the injection of the L-Aspa RBC into mice, the values obtained are at the assay detection limit (between 1 and 3 IU/litre).

### EXAMPLE 3

### Encapsulation of L-Asparaginase in Human Erythrocytes

The method described in WO-A-2006/016247 is used to produce a batch of erythrocytes encapsulating L-asparaginase. In accordance with the teaching of WO-A-2006/016247, the osmotic fragility is considered and the lysis parameters are adjusted accordingly (flow rate of the erythrocyte suspension in the dialysis cartridge is adjusted). The method is further performed in conformity with the physician prescription, which takes into account the weight of the patient and the dose of L-asparaginase to be administered. The specifications of the end product are as follows:
mean corpuscular volume (MCV): 70-95 fL
mean corpuscular haemoglobin concentration (MCHC): 23-35 g/dL
extracellular haemoglobin ≦0.2 g/dL of suspension
osmotic fragility ≦6 g/L of NaCl
mean corpuscular L-asparaginase concentration: 78-146 IU/mL
extracellular L-asparaginase ≦2% of the total enzyme activity.

The suspension of erythrocytes so obtained is called GRASPA® and is mentioned in the literature.

### COMPARATIVE EXAMPLE 4

### Typical Chemotherapy Treatment Against AML for Children and Young Adults Before 60 Years

Induction:

Aracytine 200 mg/m²/d×7 days

Mitoxantrone 12 mg/m²/d×5 days

First Consolidation:

At day 21 or later

Aracytine 3 g/m²×2/d×3 days

Amsacrine 100 mg/m²/d×3 days

Second Consolidation:

Aracytine 200 mg/m²/d×4 days

VP16 100 mg/m²/d×4 days

Daunorubicine 40 mg/m²/d×4 days

Third Consolidation:

Aracytine 3 g/m²×2/d at D1, D2, D8, D9

L-asparaginase (free form) 6000 IU/m²/d at D2, D9

### COMPARATIVE EXAMPLE 5

### Typical Chemotherapy Treatment Against AML for Unfit Patients

Those patients are treated with aracytine and/or other drugs, a palliative treatment. L-asparaginase is not used in those patients because unfit patients can not tolerate the enzyme.

### EXAMPLE 6

### Treatment According to the Invention for any Patient, Including Unfit Patients, Including Elderly; Induction Phase

1^{st} 28 days period

Cytarabine (Ara-C) 20 mg/m² bid (twice a day) at D1 to D10, daily

GRASPA® (erythrocytes encapsulating asparaginase, in suspension) 100 IU/kg at D11

2^{nd} 28 days period until 12^{th} month

Cytarabine (Ara-C) 20 mg/m² bid at D1 to D10, daily

GRASPA® 100 IU/kg at D1

### EXAMPLE 7

### Treatment According to the Invention for Unfit Patients, Including Elderly

The induction phase of Example 6 is followed in remission patients by a monthly treatment until complete recovery or until death, with:

Cytarabine (Ara-C) 20 mg/m² bid at D1 to D10, daily

GRASPA® 100 IU/kg at D1

### EXAMPLE 8

### Treatment According to the Invention for Children and Adults

The induction phase of Example 6 is followed by consolidation phases, typically 2 or 3 consolidation phases.

Preferably, GRASPA® 100 IU/kg is used at any or at some consolidation phases, along with another chemotherapy agents. In an embodiment, GRASPA® 100 IU/kg is used at all the consolidation phases.

### EXAMPLE 9

### Treatment for Children and Adults with High Dose Aracytine; Induction Phase

### 1^{st} Embodiment

Aracytine 200 mg/m²/d×7 days

Mitoxantrone 12 mg/m²/d×5 days

One dose GRASPA® 100 IU/kg at D1

### 2^{nd} Embodiment

Aracytine 200 mg/m²/d×7 days

Mitoxantrone 12 mg/m²/d×5 days

One dose GRASPA® 100 IU/kg at D1

### EXAMPLE 10

### Consolidation after Induction Phase According to Example 9

First Consolidation:

At day 21 or later

Aracytine 3 g/m²×2/d×3 days

Amsacrine 100 mg/m²/d×3 days

One dose GRASPA® 100 IU/kg

Second Consolidation:

Aracytine 200 mg/m²/d×4 days

VP16 100 mg/m²/d×4 days

Daunorubicine 40 mg/m²/d×4 days

One dose GRASPA® 100 IU/kg

Third Consolidation:

Aracytine 3 g/m²×2/d at D1, D2, D8, D9

One dose GRASPA® 100 IU/kg

1-29. (canceled)

30. A method for treating Acute Myeloid Leukemia (AML) comprising administering to a patient in need thereof an efficient amount of a suspension of erythrocytes encapsulating asparaginase, wherein one administers at least two doses of suspension during a phase treatment, wherein one dose of suspension comprises from 50 to 500 IU of encapsulated asparaginase per kg body weight and wherein two doses administered to the same patient are administered with a lag time which is above or equal to 14 days.

31. The method of claim 30, wherein two doses administered to the same patient are administered with a lag time which is from 14 to 45 days.

32. The method of claim 30, wherein one dose of suspension comprises from 50 to 200 IU of encapsulated asparaginase per kg body weight.

33. The method of claim 30, wherein one dose of suspension comprises from 80 to 170 IU of encapsulated asparaginase per kg body weight.

34. The method of claim 30, wherein one dose of suspension comprises 100 IU of asparaginase per kg body weight.

35. The method of claim 30, wherein one dose of suspension comprises 150 IU of asparaginase per kg body weight.

36. The method of claim 30, wherein the patient is a child, an adult or an elderly.

37. The method of claim 30, wherein the patient is an elderly over 65 years.

38. The method of claim 30, wherein the patient is an unfit patient.

39. The method of claim 30, wherein the suspension of erythrocytes encapsulating asparaginase is used within a chemotherapeutic protocol in which one or several other chemotherapeutic agents are used in the same patient.

40. The method of claim 39, wherein the other chemotherapeutic agent is selected from the group consisting of cytarabine, mitoxantrone, amsacrine, etoposide, thioguanine, prednisolone, vincristine, VP16, daunorubicine, azacitidine and decitabine.

41. The method of claim 39, wherein the other chemotherapeutic agent is cytarabine.

42. The method of claim 39, wherein cytarabine is used at a low dose regimen of 1 to 100 mg/m²/d.

43. The method of claim 39, wherein cytarabine is used at a low dose regimen of 5 to 50 mg/m²/d.

44. The method of claim 39, wherein cytarabine is used at a low dose regimen of 20 mg/m²/d.

45. The method of claim 39, wherein cytarabine is used at a low dose regimen of 40 mg/m²/d.

46. The method claim 39, comprising the following phase scheme:
1^{st} 28 days period
Cytarabine 40 mg/m² D1 to D10, daily
Suspension of erythrocytes encapsulating asparaginase 100 IU/kg at D11
2^{nd} 28 days period until 12^{th} month
Cytarabine 40 mg/m² at D1 to D10, daily
Suspension of erythrocytes encapsulating asparaginase 100 IU/kg at D1.
